(19) United States
(12) Patent Application Publication
(10) Pub. No.: US 2026/0283601 A1
(43) Pub. Date: Sep. 24, 2026

(54) METHOD AND SYSTEM OF ACQUISITION OF A SIGNAL REPRESENTATIVE OF THE DIAMETER OF A BLOOD VESSEL

(71) Applicant: Commissariat à l'Energie Atomique et aux Energies Alternatives, Paris (FR)

(72) Inventors: Augustin VERNAY, GRENOBLE (FR); Guillaume BLANQUER, GRENOBLE (FR); Mathieu PERRIOLLAT, GRENOBLE (FR); Pierre BLANDIN, GRENOBLE (FR); Rémi GERBELOT BARILLON, GRENOBLE (FR)

(21) Appl. No.: 19/565,905

(22) Filed: Mar. 13, 2026

(30) Foreign Application Priority Data
Mar. 20, 2025 (FR) 2502838

(51) Int. Cl. A61B 8/00

(52) U.S. Cl. CPC A61B 8/58

(57) ABSTRACT

The present description relates to a method for acquiring a signal, comprising a calibration phase comprising: 1) determining a reference signal representative of the evolution of the diameter of the blood vessel, by means of an ultrasonic sensor; 2) acquiring an optical signal by means of a photoplethysmography sensor; 3) determining, from the optical signal and the reference signal, a mathematical model adapted to generate, from the only optical signal, an estimated signal representative of the evolution of the diameter of the blood vessel during the calibration phase, the method further comprising, after the calibration phase: 4) measuring an optical signal representative of the evolution of the blood vessel, by means of the photoplethysmography sensor; and 5) calculating an estimated signal representative of the evolution of the diameter of the blood vessel, based on the signal measured in the step 4) and the mathematical model.

### TECHNICAL FIELD

The present disclosure generally relates to measurements of cardiovascular parameters and, in particular, a method and a system for acquiring a signal representative of the evolution of a diameter of a blood vessel by means of an optical sensor.

### BACKGROUND ART

Measuring an evolution of a diameter or of a characteristic quantity of a diameter of a blood vessel, of an artery for example, over time allows to monitor cardiovascular parameters such as blood pressure, heart rate, hemoglobin oxygen pulsed saturation (SpO2), arterial stiffness, pulse, etc. These parameters can be used for medical monitoring, physical and sport performance monitoring, etc.

Blood pressure is commonly measured by a blood pressure monitor comprising an inflatable cuff. However, such a cuff is cumbersome for the user and can interfere with the measurement, for example during a continuous use, particularly overnight.

It is known to measure the diameter of a blood vessel from images obtained by means of an ultrasonic sensor. However, ultrasonic imaging has disadvantages. In particular, it requires a complex acquisition chain and a significant computing power, which makes the solution difficult to miniaturize. Moreover, ultrasound imaging involves a relatively high power consumption, the generation of a large volume of data and the need to use an acoustic coupling material to obtain images of a sufficient quality.

Besides, it is known to measure a heart rate or a pulse by means of an optical sensor, also known as a photoplethysmography sensor, which can be integrated into a wearable device, for example a watch worn on the user's wrist. Such sensors are relatively bit cumbersome and require limited energy and computing resources. However, they do not allow a reliable and accurate measurement of the evolution over time of a diameter or of a characteristic quantity of a diameter of a blood vessel diameter.

There is a need to at least partially overcome some of the disadvantages of known solutions.

### SUMMARY OF INVENTION

One embodiment provides a method for acquiring a signal representative of a diameter of a blood vessel, comprising a calibration phase, the calibration phase comprising the following steps:
1) determining a reference signal representative of the evolution of the diameter of the blood vessel during a calibration period, by means of an ultrasonic sensor;
2) acquiring an optical signal, representative of the evolution of the blood vessel during the calibration period, by means of a photoplethysmography sensor;
3) determining, from the optical signal measured in the step 2) and the reference signal determined in the step 1), a mathematical model adapted to generate, from the only optical signal measured in the step 2), an estimated signal representative of the evolution of the diameter of the blood vessel during the calibration period, the method further comprising, after the calibration phase, the following steps:
4) measuring an optical signal representative of the evolution of the blood vessel during an acquisition period, by means of the photoplethysmography sensor; and
5) calculating an estimated signal representative of the evolution of the diameter of the blood vessel during the acquisition period, from the signal measured in the step 4) and the mathematical model determined in the step 3).

According to one embodiment, the step 1) comprises acquiring one or more ultrasonic signals and determining the reference signal from said ultrasonic signals.

According to one embodiment, the optical signal acquired in the step 2) comprises a plurality of optical signals acquired respectively in several acquisition configurations.

According to one embodiment, the photoplethysmography sensor comprises at least one emitter and at least one receiver, and wherein an acquisition configuration comprises an emission wavelength by the emitter and a distance between the emitter and the receiver.

According to one embodiment, in the step 3), the mathematical model is of the form:

$$\begin{matrix}[ Math 17 ] & \\ Y ( t ) = f ( \{ S ( t ) \} , p ) & \end{matrix}$$

where Y denotes the estimated signal, t denotes a time variable, f denotes a determined function, S denotes the optical signal acquired in the step 2) and p denotes a parameter vector comprising one or more parameters to be determined.

According to one embodiment, the function f is a linear function, the parameter vector p of the linear function being determined by linear regression.

According to one embodiment, said linear regression is a regularized linear regression, for example a Lasso regression.

According to one embodiment, the function f is defined as follows:

$$\begin{matrix}[ Math 18 ] & \\ f ( \{ S ( t ) \} , p ) = A * ln ( S ( t ) / {S}_{0} ) + B & \end{matrix}$$

where S₀ denotes the intensity of a wave emitted by the photoplethysmography sensor in the step 2) and A and B are two parameter values of the parameter vector p.

According to one embodiment:

$$\begin{matrix}[ Math 19 ] & \\ A = \frac{( y 1 - y 2 )}{ln ( \frac{s 1}{s 2} )} & \end{matrix}$$

$$and$$

$$\begin{matrix}[ Math 20 ] & \\ B = {y}_{1} - \frac{{y}_{1} - {y}_{2}}{ln ( \frac{{s}_{1}}{{s}_{2}} )} ln ( \frac{{s}_{1}}{{s}_{0}} ) & \end{matrix}$$

where y1 and y2 denote two values of the quantity representative of the diameter of the blood vessel determined in the step 3) and s1 and s2 denote two values of the optical signal measured in the step 2), y1 being obtained from a first measurement synchronous with s1 and y2 being obtained from a second measurement synchronous with s2.

According to one embodiment, the function f is defined by numerical simulation.

According to one embodiment, a supervised learning is used to determine the function f.

According to one embodiment, the blood vessel is an artery.

An embodiment provides a system for acquiring a signal representative of a diameter of a blood vessel comprising:
an ultrasonic sensor;
a photoplethysmography sensor; and
a data processing device configured to, during a calibration phase:
1) determining a reference signal representative of the evolution of the diameter of the blood vessel during a calibration period, by means of the ultrasonic sensor;
2) acquiring an optical signal representative of the evolution of the blood vessel during the calibration period, by means of the photoplethysmography sensor;
3) determining, from the optical signal measured in the step 2) and the reference signal determined in the step 1), a mathematical model adapted to generate, from the only optical signal measured in the step 2), an estimated signal representative of the evolution of the diameter of the blood vessel during the calibration period, the data processing device being further configured to perform, after the calibration phase, the following steps:
4) measuring an optical signal representative of the evolution of the blood vessel during an acquisition period, by means of the photoplethysmography sensor; and
5) calculating an estimated signal representative of the evolution of the diameter of the blood vessel during the acquisition period, from the signal measured in the step 4) and the mathematical model determined in the step 3).

### BRIEF DESCRIPTION OF DRAWINGS

The foregoing features and advantages, as well as others, will be detailed in the following description of particular embodiments made on a non-limiting basis with reference to the attached drawings, in which:

FIG. 1A and FIG. 1B schematically illustrate an acquisition of a signal by an optical sensor according to one embodiment;

FIG. 1C schematically illustrates an example of the evolution of a photoplethysmography signal according to one embodiment;

FIG. 2 schematically illustrates, in the form of blocks, an example of a system for acquiring a signal representative of a diameter of a blood vessel according to one embodiment; and

FIG. 3 schematically illustrates, in the form of blocks, an example of a method for acquiring a signal representative of a diameter of a blood vessel according to one embodiment.

### DESCRIPTION OF EMBODIMENTS

The same elements have been designated by the same references in the various Figures. In particular, the structural and/or functional elements common among the various embodiments may have the same references and may dispose identical structural, dimensional and material properties.

For the sake of clarity, only the steps and elements useful for an understanding of the described embodiments have been illustrated e detailed. In particular, the acquisition of signals representative of the evolution of a blood vessel by means of an ultrasonic sensor or from an optical sensor is known to those skilled in the art. Thus, these acquisitions will not be detailed in the present description. Similarly, the implementation of ultrasonic or optical sensors will not be detailed, the described embodiments being compatible with known ultrasonic or optical sensors, or the implementation of these sensors being within the reach of those skilled in the art based on the indications of the present description. Furthermore, the determination of a characteristic quantity of the diameter of a blood vessel from one or more ultrasonic signals is also known to those skilled in the art and will not be detailed below. In addition, the implementation of the control and processing electronic units or circuits of the described systems has not been detailed, this being within the reach of those skilled in the art based on the functional indications of the present description.

Unless otherwise specified, when reference is made to two elements connected together, this signifies a direct connection without any intermediate elements other than conductors, and when reference is made to two elements connected or coupled together, this signifies that these two elements can be connected or coupled via one or more other elements.

In the following disclosure, unless otherwise specified, when reference is made to absolute positional qualifiers, such as the terms “front”, “back”, “top”, “bottom”, “left”, “right”, etc., or to relative positional qualifiers, such as the terms “above”, “below”, “higher”, “lower”, etc., or to qualifiers of orientation, such as “horizontal”, “vertical”, etc., reference is made to the orientation of the Figures in a normal position of use.

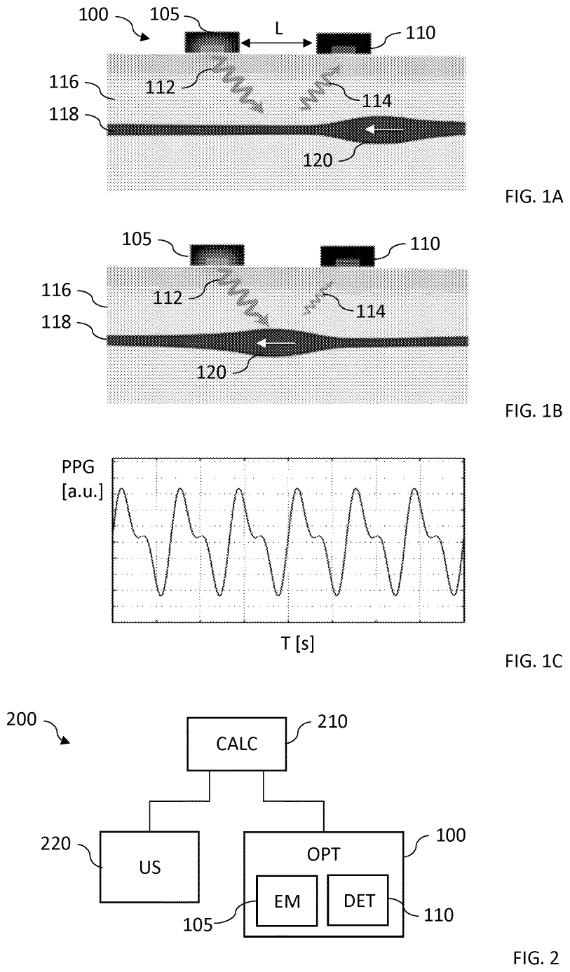

FIG. 1A and FIG. 1B schematically illustrate an acquisition of a signal by an optical sensor 100 according to one embodiment.

The optical sensor 100 is, for example, a photoplethysmography sensor.

In the example of FIGS. 1A and 1B, the optical sensor 100 comprises an emitter 105 and a receiver 110. The emitter 105, for example a diode, is configured to emit an incident optical wave 112, for example a wave having a wavelength in the visible or infrared or near-infrared spectrum. The receiver 110, for example a photodiode, is configured to detect a portion of the incident optical wave 112 that is reflected or backscattered 114.

As an example, the emitter 105 and the receiver 110 are positioned in contact with and on the surface of the skin of a user, for example an animal or a human being, for example opposite a blood vessel 118, for example a vein or preferably an artery. The incident wave 112 is, for example, at least partially transmitted by tissues 116, for example comprising one or more layers of skin, one or more muscles, etc., before reaching the blood vessel 118. The incident wave 112 is, for example, at least partially backscattered by the encountered media. The backscattered wave 114 is detected by the receiver 110 of the optical sensor 100. The intensity of the reflected wave 114 is lower than an intensity of the incident wave 112.

As a variant, an acquisition of a signal by a photoplethysmography sensor can be performed in an emission mode, i.e. the emitter 105 and the receiver 110 are located on either side of the area to be probed, for example the emitter 105 is above the area to be probed and the receiver 110 is below the area to be probed. This embodiment is suitable, for example, for relatively thin regions, for example a finger or an earlobe, allowing a part of the radiation to pass through the area to be probed.

In the illustrated example, a pulse wave 120, illustrated by an arrow in FIG. 1A and FIG. 1B, travels in the blood vessel 118.

A diameter of the blood vessel 118 evolves during the propagation of the pulse wave. The wavelength of the incident optical wave 112 is selected, for example, to be absorbed more strongly by blood than by surrounding tissues. When the diameter of the blood vessel 118 increases opposite the optical sensor 100, the volume of blood inside the vessel increases locally, the incident wave 112 is, for example, relatively more absorbed the and intensity of the backscattered wave 114, measured by the receiver 110, decreases. In other words, an increase of the blood volume leads to an increase of the absorption and, consequently, a decrease of the intensity of the backscattered wave 114.

The selection of the used wavelength(s) influences, for example, the amplitude of an evolution of the signal measured by the receiver 110.

The optical sensor 100 can be used regardless of the orientation of the artery relative to the positions of the emitter 105 and of the receiver 110.

The emitter 105 and the receiver 110 are, for example, separated by a distance L. The distance L is, for example, configured to discriminate backscattered rays having different trajectories and/or to discriminate a depth of the blood vessel 118 being optically probed.

FIG. 1C schematically illustrates an example of the evolution of a photoplethysmography signal (“PPG [a.u.]”) over time (“T[s]”) according to one embodiment.

The photoplethysmography signal is obtained, for example, by means of the optical sensor 100 described in relation to FIGS. 1A and 1B. The photoplethysmography signal corresponds, for example, to an intensity of the backscattered wave 114. An evolution of the photoplethysmography signal is correlated, for example, with an evolution of the blood volume in the probed area, which can be linked to an evolution of the diameter of the blood vessel 118. Such a signal is typically used to measure the user's heart rate, but does not directly allow to go back to the evolution over time of a value of the diameter of the blood vessel 118.

According to one embodiment of the present description, an ultrasonic sensor is used during a calibration phase to establish a relationship between the evolution of the signal obtained by the optical sensor 100 and the evolution of the diameter of the blood vessel 118. During the calibration phase, a mathematical model is defined, allowing to generate, from the only optical signal, a signal representative of the evolution over time of a value representative of the diameter of the blood vessel. At the end of the calibration phase, a signal representative of the evolution over time of a value representative of the diameter of the blood vessel is generated based on the only measurements provided by the optical sensor.

FIG. 2 schematically illustrates, in the form of blocks, an example of a system 200 for acquiring a signal representative of a diameter of a blood vessel according to one embodiment.

The system 200 comprises a data processing device 210 (“CALC”), an ultrasonic sensor 220 (“US”) and an optical sensor 100 (“OPT”). The optical sensor 100 corresponds, for example, to the optical sensor described in relation to FIGS. 1A and 1B. The optical sensor 100 comprises, for example, the emitter 105 (“EM”) and the detector 110 (“DET”). The ultrasonic sensor 220 and the optical sensor 100 are, for example, coupled to the data processing device 210.

The ultrasonic sensor 220 comprises one or more ultrasonic transducers, for example, a linear array or a matrix of ultrasonic transducers.

The ultrasonic sensor 220 is configured to generate an ultrasonic wave incident in the tissues 116 and to detect an ultrasonic wave reflected or backscattered by the tissues 116 and the blood vessel 118, so as to generate signals representative of the evolution of the structure of the blood vessel 118. These signals are, for example, reconstructed in the form of one or more images, or are, for example, interpreted in terms of echoes to locate various interfaces in the tissues, and in particular the walls of the artery.

The ultrasonic sensor 220 and the optical sensor 100 are configured, for example, to take measurements of the same blood vessel 118, for example at a same position or at relatively close positions, for example distant by a distance of less than or equal to 1 cm, preferably less than or equal to 5 mm. As an example, the ultrasonic sensor 220 and the optical sensor 100 are configured to take synchronous or simultaneous measurements during the calibration phase. For example, a temporal readjustment is performed by the data processing device 210 to take into account a distance between the sensors.

The ultrasonic sensor 220 and the optical sensor 100 are configured, for example, to transmit the measurements to the data processing device 210. The data processing device 210 is configured, for example, to analyze the measurements. The data processing device 210 comprises, for example, at least one microprocessor and at least one memory circuit.

According to one embodiment, the data processing device 210 is configured to establish a relationship between a characteristic quantity of the diameter of the blood vessel 118, obtained from an ultrasonic measurement, and an optical measurement.

According to one embodiment, the data processing device 210 comprises two computing units: a first computing unit configured to perform a calibration of the system 200 and a second computing unit configured to use the calibration on one or more measurements transmitted by the ultrasonic sensor 220 and/or the optical sensor 100. The first computing unit is, for example, configured to have a relatively large computing capacity and the second computing unit is, for example, configured to be less cumbersome and/or to consume less energy than the first computing unit. As a variant, the calibration and the measurements are performed by a same computing unit.

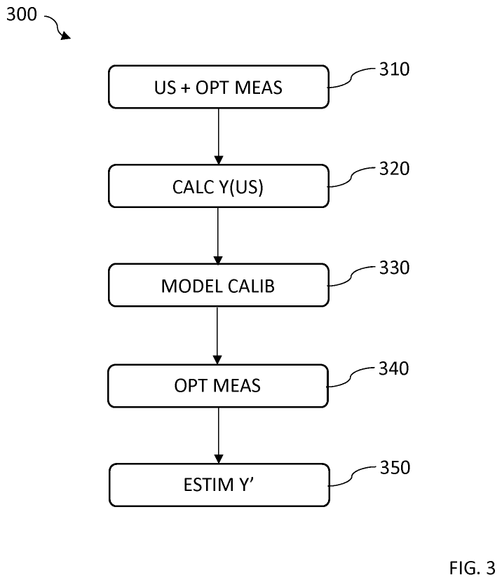

FIG. 3 schematically illustrates, in the form of blocks, an example of a method 300 for acquiring a signal representative of a diameter of a blood vessel according to one embodiment. The method 300 is, for example, implemented by means of the system 200 of FIG. 2.

The method 300 comprises a step 310 (“US+OPT MEAS”) for measuring optical and ultrasonic signals representative of an evolution over time of the blood vessel 118 during a calibration period.

The ultrasonic sensor 220, described in relation to FIG. 2, is for example configured to, during the step 310, acquire one or more ultrasonic signals of a same region of the blood vessel 118 during the calibration period. The ultrasonic signals are, for example, acquired successively, for example with an acquisition frequency between 10 Hz and 1 kHz, and for example between 10 Hz and 500 Hz.

The optical sensor 100, described in relation to FIGS. 1A, 1B and 2, is for example configured to, during the step 310, perform one or more measurements of optical signals representative of the evolution of the same region of the blood vessel 118 during the same calibration period. The ultrasonic sensor 220 and the optical sensor 100 are configured, for example, to perform synchronous measurements.

As an example, the optical sensor 100 is configured to perform one or more successive measurements for each of one or more acquisition configurations c. An acquisition configuration c is, for example, defined by a wavelength of the incident wave 112 and a distance L between the emitter 105 and the detector 110 or is defined, for example, by a wavelength of the incident wave 112 and a pair comprising an emitter 105 and a detector 110 from among a plurality of emitters and/or receivers. Each acquisition configuration c is, for example, applied simultaneously or successively, within a short time interval compared to the acquisition frequency.

Each pair comprising an emitter and a detector is, for example, positioned in a relevant manner to probe an area of interest.

According to one embodiment, several wavelengths are used by the optical sensor. The tissues 116 comprise, for example, different elements, for example a dermis, a hypodermis, one or more muscles, etc. The different elements have, for example, different compositions and/or properties, in particular different optical properties. The use of several wavelengths makes it possible, for example, to discriminate variations of the backscattered wave 114 caused by the blood vessel 118 and variations caused by the tissues 116. For example, a spectral analysis is performed with at least two wavelengths. Moreover, the absorption by the tissues 116 varies with the wavelength of the incident wave 112. The wavelength(s) is or are configured, for example, to probe a volume, for example a fixed volume, of tissues. For example, a first wavelength is used for a first acquisition to probe superficial tissues and a second wavelength is used for a second acquisition to probe superficial and deep tissues as well as the blood vessel(s) 118. For example, an operation is performed between the signal from the first acquisition and the signal from the second acquisition to remove the disturbances induced by the superficial tissues. For example, the signal from the first acquisition is subtracted from the signal from the second acquisition. The optical sensor 100 comprises, for example, several emitters 105 and/or several receivers 110. For example, each emitter 105, for example a diode, is configured or selected to emit at a specific or fixed wavelength and each receiver 110, for example a photodiode, is configured to detect a range of wavelengths or a wave having a fixed wavelength, for example by means of one or more filters. The emitters 105 are, for example, relatively close to each other in order to probe the same blood vessel 118.

According to another embodiment, several distances L are used, for example for a wavelength or for several wavelengths. A fixed tissue volume is, for example, associated with the distance L. For example, the receiver 110 comprises several detection elements, for example several photodiodes, each detection element having a fixed distance from the emitter 105.

According to one embodiment, at least two acquisition configurations c are applied, for example for two wavelengths and one distance or for two distances and one wavelength. As an example, acquisitions are performed for several wavelengths and/or several distances, sequentially or simultaneously. A large number of acquisition configurations has the advantage of increasing a collected informational content in order to better extract a signal of interest.

For example, the system 200 is configured so that the optical sensor 100 records signals in m acquisition configurations, denoted c1, . . . , cm, during the calibration period, where m corresponds to:

$$\begin{matrix}[ Math 1 ] & \\ m = {\sum }_{p = 1}^{k} {L}_{p} & ( 1 )\end{matrix}$$

where k denotes the number of distinct wavelengths for which signals are recorded, with k an integer greater than or equal to 1, preferably greater than or equal to 2, and p is an integer ranging from 1 to k. Lₚ denotes the number of distinct distances L between the emitters 105 and their detectors 110 for the wavelength of rank p, with Lₚ integers greater than or equal to 1, preferably greater than or equal to 2. The system 200 is for example further configured so that the optical sensor 100 records signals for n time steps or time intervals, denoted t1, . . . , tn, during the calibration period, with n an integer greater than or equal to 1, for example strictly greater than 1, for example greater than or equal to 10, for example greater than or equal to 100. For each time step, the optical sensor 100 performs, for example, m measurements, corresponding, for example, to one measurement per acquisition configuration. The n time steps are, for example, evenly distributed throughout the calibration period, or are, for example, unevenly distributed throughout the calibration period. As a non-limiting example, the ultrasonic sensor 220 is configured to acquire ultrasonic signals at the same n time steps as the optical sensor throughout the calibration phase.

The ultrasonic sensor 220 and the optical sensor 100 are configured, for example, to transmit the signals measured during or at the end of the calibration period to the data processing device 210.

The method 300 further comprises, after the step 310, a step 320 (“CALC Y (US)”) of determining a signal Y representative of the evolution of a diameter of the blood vessel 118 during the calibration period, based on the signals acquired by the ultrasonic sensor 220 during the calibration period.

As an example, the data processing device 210 determines a calibration vector \tilde{Y}ₙ defined by:

$${\tilde{Y}}_{n} = ( \begin{matrix}y 1 \\ ⋮ \\ yn\end{matrix} ) ,$$

where y1, . . . , yn correspond respectively to values representative of a characteristic quantity of the diameter of the blood vessel 118 determined from the ultrasonic measurements for each of the n time steps of the calibration period. In particular, yj or y(tj) denotes the value representative of a characteristic quantity of the diameter of the blood vessel 118 determined for the time step tj, with j an integer ranging from 1 to n.

By convention, in the present description, the values named with a tilde illustrate calibration data.

As an example, the blood vessel 118 is not cylindrical and the characteristic quantity of the diameter of the blood vessel 118 corresponds to the surface area of a section of the blood vessel 118 or to an average diameter of the section of the blood vessel 118 or to a height of the blood vessel 118 in a vertical plane or to the diameter of a circle having the same surface area as that of the section of the vessel in question, or to any other characteristic dimension of the section of the blood vessel. For example, the characteristic quantity Y of the diameter of the blood vessel 118 corresponds to the diameter of a circle having the surface area A of the section of the blood vessel 118 such that:

$$Y ( t ) = 2 \sqrt{\frac{A ( t )}{π}}$$

where t is a time variable.

The determination of a characteristic quantity of the diameter of a blood vessel from measurements performed by means of an ultrasonic sensor is known to those skilled in the art and will not be further detailed.

The method 300 further comprises, after the steps 310 and 320, a step 330 (“MODEL CALIB”) of determining, by the data processing device 210, a mathematical model for generating, from the only optical signal, a signal Y representative of the evolution over time of a value representative of the diameter of the blood vessel. As an example, the step 330 comprises the calculation of a parameter vector p comprising at least two calibration coefficients of the model by the data processing device 210.

The calibration coefficients are calculated from the n characteristic quantities of the diameter of the blood vessel 118 determined in the step 320 from the ultrasound measurements and from the n*m values of optical signals measured in the step 310.

The n*m optical values of optical signals measured in the step 310 are collected in a calibration matrix \tilde{S}_{m×m} defined by:

$${\tilde{S}}_{nxm} = ( \begin{matrix}\tilde{s} 11 & … & \tilde{s} 1 m \\ ⋮ & ⋱ & ⋮ \\ \tilde{s} n 1 & … & \tilde{s} nm\end{matrix} )$$

where \tilde{s}ij denotes a signal measured by means of the optical sensor 100 for the acquisition configuration cj and at the time step ti, with j an integer ranging from 1 to m.

According to one embodiment, the mathematical model is defined with a function f such that:

$$\begin{matrix}[ Math 2 ] & \\ Y ( t ) = f ( \{ {S}_{c} ( t ) \} , p ) & ( 2 )\end{matrix}$$

where t is a time variable, S_{c} corresponds to the signal measured by the optical sensor 220 for the acquisition configuration c, i.e. S_{c} corresponds to the vector

$$( \begin{matrix}sc 1 \\ ⋮ \\ scn\end{matrix} ) = ( \begin{matrix}sc ( t 1 ) \\ ⋮ \\ sc ( tn )\end{matrix} )$$

and p denotes the parameter vector.

According to a first embodiment, a linear model is used and a linear regression is performed by the data processing device 210 to determine the calibration coefficients.

The linear model is defined by:

$$\begin{matrix}[ Math 3 ] & \\ Y = p 0 + {S}_{nxm} * {P}_{m} & ( 3 )\end{matrix}$$

where:
S_{n×m} is a matrix comprising values of optical signals;
p0 is a calibration coefficient; and
Pₘ corresponds to the vector

$$( \begin{matrix}p 1 \\ ⋮ \\ pm\end{matrix} )$$

where p0, . . . , pm are calibration coefficients.

For example,

$$p = ( \begin{matrix}p 0 \\ {P}_{m}\end{matrix} )$$

is noted.

Performing a linear regression to solve the equation (2) is equivalent to solving the following system:

$$\begin{matrix}[ Math 4 ] & \\ {arg min}_{p} ‖ p 0 + {\tilde{S}}_{nxm} * {P}_{m} - {\tilde{Y}}_{n} ‖ & ( 4 )\end{matrix}$$

where:
argmin corresponds to the minimum argument of a function, i.e. the value of the parameter vector p for which the function reaches its minimum; and
∥·∥ corresponds to a norm of a vector, for example the Euclidean norm.

A least squares method is used, for example, to solve the system of the equation (4) and to determine the calibration coefficients p0, p1, . . . , pm.

In this embodiment, of the number of the sum acquisition time steps n and of the number of acquisition configurations m is greater than or equal to three. For example, n is greater than or equal to 1 and m is greater than or equal to 2. As a variant, n is greater than or equal to 2 and m is greater than or equal to 1.

According to a second embodiment, a regularized linear regression, for example by means of the Lasso method, is performed by the data processing device 210 to determine the calibration coefficients. The model is also defined by the equation (3).

The calibration coefficients are determined, for example, by solving the following system by means of the Lasso cost function:

$$\begin{matrix}[ Math 5 ] & \\ {arg min}_{p} [ \frac{1}{2 n} ‖ p 0 + {\tilde{S}}_{nxm} * {P}_{m} - {\tilde{Y}}_{n} ‖ + λ {\sum }_{j = 1}^{m} \vert {p}_{j} \vert ] & ( 5 )\end{matrix}$$

where:
λ is a regularization parameter; and
∥·∥ corresponds to the norm of a vector, for example the Euclidean norm.

A non-linear optimization method is used, for example, to solve the equation (5) and to determine the calibration coefficients p0, p1, . . . , pm.

In this embodiment, the sum of the number of acquisition time steps n and of the number of acquisition configurations m is greater than or equal to three.

The larger the regularization parameter A is, the larger the term

$$λ {\sum }_{j = 1}^{m} \vert {p}_{i} \vert$$

of the equation (5) is, and the more the solution will tend to set certain calibration coefficients to zero. As an example, during a first calibration phase, the equation (5) is solved for several values of A. Thus, it is possible, for example, to determine a number of acquisition configurations m to be performed and combined in order to preserve the quality of the calibration phase and to reduce the number of acquisition configurations to be performed during subsequent calibration phases. One advantage of this method is a reduction of the duration of the calibration phase and a reduction of the calculation time associated with the determination of a smaller number of acquisition configurations m.

According to a third embodiment, a physical model, for example based on an approximation of the Beer-Lambert equation, is used to model a variation of the intensity of the optical signal as an output from the tissues 116. As an example, the tissues 116 are considered to be homogeneous with an effective attenuation coefficient Jeff that is constant. The equation of the intensity of the optical signal S_{cj} measured by the optical sensor 100 is given by:

$$\begin{matrix}[ Math 6 ] & \\ {S}_{cj} ( t ) = {S}_{cj}^{0} {e}^{- {μ}_{eff} \cdot {L}_{t}} {e}^{- {μ}_{art} \cdot {K}_{j} {Y}_{j} ( t )} & ( 6 )\end{matrix}$$

where:

$${S}_{cj}^{0}$$

corresponds to the light intensity of the incident wave 112 as an output of the emitter 105 of the optical sensor 100, in the acquisition configuration cj;
μ_{eff} corresponds to the effective attenuation coefficient taking into account the energy loss due to the absorption and to the scattering of the light in the part of the medium considered to be static;
Lₜ corresponds to a characteristic interaction length traveled by light in the tissues 116;
Yⱼ corresponds to the signal Y, determined, in this embodiment, from the measurements of optical signals acquired during the step 310 for the acquisition configuration cj;
μₐᵣₜ corresponds to the effective attenuation coefficient taking into account the diffusion and the absorption in the blood vessel 118; and
Kⱼ is a fixed coefficient given for a configuration cj linking a characteristic interaction length of the light in the blood vessel 118 to the characteristic quantity of the diameter of the blood vessel 118 Yⱼ.

The product Kⱼ·_{Yj} corresponds, for example, to a characteristic interaction length of the light in the blood vessel 118.

The equation (6) can be written as:

$$\begin{matrix}[ Math 7 ] & \\ {Y}_{j} ( t ) = bj + aj \cdot ln ( \frac{{S}_{cj} ( t )}{{S}_{cj}^{0}} ) & ( 7 )\end{matrix}$$

with

$$bj = - \frac{1}{{μ}_{art} \cdot {K}_{j}} and aj = - \frac{{μ}_{eff} \cdot {L}_{t}}{{μ}_{art} \cdot {K}_{j}} \cdot {D}_{cj} = ln ( \frac{{S}_{cj} ( t )}{{S}_{cj}^{0}} )$$

$$and {\tilde{D}}_{cj} = ln ( \frac{{\tilde{S}}_{cj}}{{\tilde{S}}_{cj}^{0}} ) with {\tilde{S}}_{cj} = ( \begin{matrix}si 1 \\ ⋮ \\ sin\end{matrix} ) = ( \begin{matrix}{S}_{cj} ( t 1 ) \\ ⋮ \\ {S}_{cj} ( tn )\end{matrix} )$$

are noted.

Lₜ corresponds to a characteristic interaction length of the optical wave with the tissues 116.

For example,

$$p = ( \begin{matrix}aj \\ bj\end{matrix} )$$

is noted.

As an example, the coefficients aj and bj are for example determined by means of two pairs of values {S_{c1}(t1), y(t1)} and {S_{c1}(t2), y(t2)}.

More than two measurements are for example performed for a same acquisition configuration c1 and the pair of values {S_{c1}(t1), y(t1)} corresponds to the pair whose the intensity S_{c1}(t1) is maximum and the pair of values {S_{c1}(t2), y(t2)} corresponds to the pair whose the intensity S_{c1}(t2) is minimum.

The constant aj and the constant bj are determined, for example, by solving a linear equation with two unknowns, for which at least two measurements are used, or, for example, by using a least-squares method.

The constant aj is equal to:

$$\begin{matrix}[ Math 8 ] & \\ aj = \frac{y 1 - y 2}{ln ( \frac{s 1}{s 2} )} & ( 8 )\end{matrix}$$

with y1=y(t1), y2=y(t2), s1=S_{c1}(t1) and s2=S_{c1}(t2).

The constant bj is equal to:

$$\begin{matrix}[ Math 9 ] & \\ bj = {y}_{1} - \frac{{y}_{1} - {y}_{2}}{ln ( \frac{{s}_{1}}{{s}_{2}} )} ln ( \frac{{s}_{1}}{{s}_{0}} ) & ( 9 )\end{matrix}$$

with y1=y(t1), y2=y(t2), s1=S_{c1}(t1) and s2=S_{c1}(t2).

According to another example, when n is different from two, the calibration coefficients are determined by solving:

$$\begin{matrix}[ Math 10 ] & \\ {arg min}_{p} ‖ aj * {\tilde{D}}_{cj} + bj - {\tilde{Y}}_{n} ‖ & ( 10 )\end{matrix}$$

A least squares method is used, for example, to solve the system of the t equation (10) and to determine the calibration coefficients aj and bj.

In this embodiment, the number of acquisition time steps n is greater than two and the number of acquisition configurations m is greater than or equal to one.

According to a fourth embodiment, the previous method is repeated for each acquisition configuration ci.

As an example, the signal Y corresponds to the average of the signals Yᵢ determined by means of the previous method for each acquisition configuration ci.

D_{n×m} denotes a matrix obtained by concatenating the vectors D_{ci} and \tilde{D}_{(n×m)} denotes a matrix obtained by concatenating the vectors \tilde{D}_{cj}.

$${A}_{m} = ( \begin{matrix}a 1 \\ ⋮ \\ am\end{matrix} ) and {B}_{m} = ( \begin{matrix}b 1 \\ ⋮ \\ bm\end{matrix} )$$

are denoted.

The model is thus defined by:

$$\begin{matrix}[ Math 11 ] & \\ Y = \frac{1}{m} {D}_{nxm} * {A}_{m} + \frac{1}{m} {1}_{nxm} * {B}_{m} & ( 11 )\end{matrix}$$

where 1_{n×m} Corresponds to the identity matrix of size n×m.

$$p = ( \begin{matrix}{A}_{m} \\ {B}_{m}\end{matrix} )$$

is denoted.

The calibration coefficients are determined by solving:

$$\begin{matrix}[ Math 12 ] & \\ {arg min}_{p} ‖ \frac{1}{m} ( {\tilde{D}}_{nxm} {1}_{nxm} ) * p - {\tilde{Y}}_{n} ‖ & ( 12 )\end{matrix}$$

A least squares method, for example supplemented by a regularization of the parameters, is for example used to solve the system of the equation (12) and to determine the parameter vector p.

According to a fifth embodiment, a physical model M that is relatively complex is considered and a numerical simulation is used to determine the calibration coefficients and to solve the equation (2) based on the optical measurements performed in the step 310 and on the values of the characteristic quantity of the diameter of the blood vessel 118 determined in the step 320 of the method 300.

The physical model M is expressed, for example, in a literal form, by a numerical approach, for example by means of finite elements, or by a Monte Carlo-type simulation.

The model, for an acquisition configuration cj, is defined, for example, by:

$$\begin{matrix}[ Math 13 ] & \\ {S}_{cj} ( t ) = M ( Y ( t ) , p ) & ( 13 )\end{matrix}$$

The parameter vector p brings together, for example, parameters of the model that must be adapted to the subject, for example absorption coefficients and physiognomic quantities, for example a nominal diameter, a depth of the blood vessel 118, etc.

The calibration coefficients are determined by solving:

$$\begin{matrix}[ Math 14 ] & \\ {arg min}_{p} ‖ M ( {\tilde{Y}}_{n} , p ) - {\tilde{S}}_{nxm} ‖ & ( 14 )\end{matrix}$$

The signal Y is then determined by solving:

$$\begin{matrix}[ Math 15 ] & \\ {arg min}_{Y} ‖ M ( Y , p ) - {S}_{nxm} ‖ & ( 15 )\end{matrix}$$

According to a sixth embodiment, a supervised learning method is used.

During a learning phase of an algorithm, the synchronous data {\tilde{Y}ₙ,\tilde{S}_{n×m}} is used to train a model R. The signal Y is then determined according to:

$$\begin{matrix}[ Math 16 ] & \\ Y = R ( {S}_{nxm} , p ) & ( 16 )\end{matrix}$$

The steps 310, 320 and 330 of the method 300 correspond, for example, to a calibration phase of the method 300.

The method 300 further comprises, for example, after the calibration phase, a step 340 (“OPT MEAS”) of optical measurement by the optical sensor 100 of the system 200 during an acquisition period.

The optical sensor 100 is configured, for example, to measure optical signals S_{m×m}′ representative of an evolution of the blood vessel 118 during the acquisition period.

The method 300 further comprises, for example, after the step 340, a step 350 (“ESTIM Y′”) of determining a signal Y′ representative of the evolution of a characteristic quantity of the diameter of the blood vessel 118 by the data processing device 210 of the system 200.

The data processing device 210 uses the mathematical model determined in the step 330 and defined by the equation (2), or by one of the equations (3), (7), (11), (15) and (16), to calculate the signal Y′ from the optical measurements S_{n×m}′ performed in the step 340.

An advantage of this method is that the calibration phase comprising the steps 310, 320 and 330 is, for example, performed only once or is, for example, performed periodically, for example at a frequency between once every 20 minutes and once a week. Between calibration phases, the data processing device 210 is configured, for example, to determine a quantity representative of the diameter of the blood vessel 118 based on the only measurements performed by the optical sensor 100, according to the steps 340 and 350 of the method 300, without using the ultrasonic sensor. This makes it possible, during the acquisition phases between two successive calibration phases, to obtain reliable measurements with limited energy consumption and reduced calculation times, and a reduction of the required calculation performance.

As an example, the optical sensor 100 is integrated into a wearable accessory, for example a bracelet, a watch, a patch, an armband, etc. The data processing device 210 is, for example, integrated into the wearable accessory or is, for example, in an external device, for example a computer, a mobile phone, etc. For example, the measurements performed by the optical sensor 100 are sent, via a wired or wireless connection, for example a radio link, to the data processing device 210. The measurements are sent, for example, after each acquisition or periodically or by a manual or automatic data transfer performed by the user.

According to one embodiment, the ultrasonic sensor 220 is integrated into the accessory. An advantage of the method 300 is then a reduction of the power consumption of the accessory and a reduction of the data processing time resulting from the fact that the ultrasonic sensor 220 is used only during the calibration phases.

According to another embodiment, the ultrasonic sensor 220 is a device external to the accessory, for example a relatively bulky ultrasound scanner, for example non-wearable, which is coupled to the accessory only during the calibration phases. An additional advantage of the method 300 is then a reduction of the size of the accessory, for example resulting in less discomfort for the user and/or facilitating an acquisition throughout the day and improving the compatibility of the accessory with user's activities. Another advantage of measurements performed by means of an optical sensor is that the use of a gel is not necessary to obtain a qualitative signal.

An advantage of the presented various embodiments is the obtaining of a characteristic quantity of the diameter of a blood vessel, for example of an artery, and of its evolution over time, from measurements acquired by an optical sensor. Moreover, the detailed devices allow a measurement of a characteristic quantity of the diameter of a blood vessel in a continuous manner during daily activities, which is for example difficult with a cuff and impossible with an ultrasound scanner. A quantitative value, in a unit of length, of the characteristic quantity of the diameter is obtained and can be directly compared with measurements obtained by means of an ultrasonic sensor. Another advantage of the presented various embodiments is the obtaining of reliable values and a method adapted to the diversity of users and acquisition configurations.

One application of the detailed devices in the present description and of the method 300 is a medical monitoring, for example over an extended time period. Another application is a monitoring of the cardiac activity, for example for a sportsperson.

Measuring an evolution of the diameter of a blood vessel, for example an artery, over time allows for monitoring of cardiovascular parameters such as blood pressure, heart rate, hemoglobin oxygen pulsed saturation (SpO2), arterial stiffness, pulse, etc.

Various embodiments and variants have been described. Those skilled in the art will understand that certain features of these various embodiments and variants can be combined and other variants will readily occur to those skilled in the art. In particular, the receiver 110 of the optical sensor 100 is, for example, a matrix receiver. The calibration steps 210, 220 and 100 of the method 300 are, for example, performed for different units of the receiver in order to determine the units that allow for an optimal calibration. A quality of the calibration is, for example, determined by measuring a root mean square deviation between the optical measurements and the corresponding ultrasonic measurements performed in the step 310.

According to one embodiment, the calibration phase is performed several times by means of different resolution methods to determine the resolution method resulting in the best calibration quality. This resolution method is, for example, selected for future calibration phases.

Finally, the practical implementation of the described embodiments and variants is within the reach of those skilled in the art based on the hereinabove provided functional indications.

1. A method for acquiring a signal representative of a diameter of a blood vessel, comprising a calibration phase, the calibration phase comprising the following steps:
1) determining a reference signal representative of the evolution of the diameter of the blood vessel during a calibration period, by means of an ultrasonic sensor;
2) acquiring an optical signal representative of the evolution of the blood vessel during the calibration period, by means of a photoplethysmography sensor;
3) determining, from the optical signal measured in the step 2) and the reference signal determined in the step 1), a mathematical model adapted to generate, from the only optical signal measured in the step 2), an estimated signal representative of the evolution of the diameter of the blood vessel during the calibration period,
the method further comprising, after the calibration phase, the following steps:
4) measuring an optical signal representative of the evolution of the blood vessel during an acquisition period, by means of the photoplethysmography sensor; and
5) calculating an estimated signal representative of the evolution of the diameter of the blood vessel during the acquisition period, from the signal measured in the step 4) and the mathematical model determined in the step 3).

2. The acquisition method according to claim 1, wherein the step 1) comprises acquiring one or more ultrasonic signals and determining the reference signal from said ultrasonic signals.

3. The acquisition method according to claim 1, wherein the optical signal acquired in the step 2) comprises a plurality of optical signals acquired respectively in several acquisition configurations.

4. The acquisition method according to claim 3, wherein the photoplethysmography sensor comprises at least one emitter and at least one receiver, and wherein an acquisition configuration comprises an emission wavelength by the emitter and a distance between the emitter and the receiver.

5. The acquisition method according to claim 1, wherein, in the step 3), the mathematical model is of the form:
$$Y ( t ) = f ( \{ S ( t ) \} , p )$$
where Y denotes the estimated signal, t denotes a time variable, f denotes a determined function, S denotes the optical signal acquired in the step 2) and p denotes a parameter vector comprising one or more parameters to be determined.

6. The acquisition method according to claim 5, wherein the function f is a linear function, the parameter vector p of the linear function being determined by linear regression.

7. The acquisition method according to claim 6, wherein said linear regression is a regularized linear regression, for example a Lasso regression.

8. The acquisition method according to claim 5, wherein the function f is defined as follows:
$$f ( \{ S ( t ) \} , p ) = A * ln ( S ( t ) / {S}_{0} ) + B$$
where S₀ denotes the intensity of a wave emitted by the photoplethysmography sensor in the step 2) and A and B are two parameter values of the parameter vector p.

9. The acquisition method according to claim 8, wherein:
$$A = \frac{( y 1 - y 2 )}{ln ( \frac{s 1}{s 2} )}$$
$$and$$
$$B = {y}_{1} - \frac{{y}_{1} - {y}_{2}}{ln ( \frac{{s}_{1}}{{s}_{2}} )} ln ( \frac{{s}_{1}}{{s}_{0}} )$$
where y1 and y2 denote values of the quantity representative of the diameter of the blood vessel determined in the step 3) and s1 and s2 denote two values of the optical signal measured in the step 2), y1 being obtained from a first measurement synchronous with s1 and y2 being obtained from a second measurement synchronous with s2.

10. The acquisition method according to claim 5, wherein the function f is defined by numerical simulation.

11. The acquisition method according to claim 5, wherein a supervised learning is used to determine the function f.

12. The acquisition method according to claim 1, wherein the blood vessel is an artery.

13. A system for acquiring a signal representative of a diameter of a blood vessel comprising:
an ultrasonic sensor;
a photoplethysmography sensor; and
a data processing device configured to, during a calibration phase:
1) determining a reference signal representative of the evolution of the diameter of the blood vessel during a calibration period, by means of the ultrasonic sensor;
2) acquiring an optical signal representative of the evolution of the blood vessel during the calibration period, by means of the photoplethysmography sensor;
3) determining, from the optical signal measured in the step 2) and the reference signal determined in the step 1), a mathematical model adapted to generate, from the only optical signal measured in the step 2), an estimated signal representative of the evolution of the diameter of the blood vessel during the calibration period,
the data processing device being further configured to perform, after the calibration phase, the following steps:
4) measuring an optical signal representative of the evolution of the blood vessel during an acquisition period, by means of the photoplethysmography sensor; and
5) calculating an estimated signal representative of the evolution of the diameter of the blood vessel during the acquisition period, from the signal measured in the step 4) and the mathematical model determined in the step 3).
